(19) United States
(12) Patent Application Publication
(10) Pub. No.: US 2008/0127394 A1
(43) Pub. Date: Jun. 5, 2008

(54) PROTECTIVE GARMENTS

(76) Inventor: Tammany Paige Atkinson, Mississauga (CA)

Correspondence Address: MILLER THOMPSON, LLP, Scotia Plaza, 40 King Street West, Suite 5800, TORONTO, ON, M5H 3S1, omitted

(21) Appl. No.: 11/945,779

(22) Filed: Nov. 27, 2007

Related U.S. Application Data
(60) Provisional application No. 60/861,098, filed on Nov. 27, 2006.

(51) Int. Cl. A41D 17/00; A41D 13/00; A41D 19/01; A41D 27/20; A61F 13/15; A47G 9/00

(52) U.S. Cl. 2/22; 36/2 R; 2/158; 2/247; 604/358; 5/485; 5/482

(57) ABSTRACT

A garment having a cushion material, such as neoprene applied to be inserted into a pocket of the garment.

### FIELD OF THE INVENTION

This invention relates generally to protective garments and particularly relates to protective garments for individuals as well as infants. This invention further relates to devices used by individuals that can be utilized in the vicinity of joints, knees or other areas that are prone to bumps and bruises. More particularly this invention relates to gaiters and diaper pads for infants.

### BACKGROUND TO THE INVENTION

Protective devices are well known in the prior art and have been utilized in different context. For example, trades people such as electricians, floor tillers, and construction workers often use knee pads or knee protectors when they are engaged in activities which required kneeling for extended periods of time. Without the knee protection the kneeling can result in much discomfort and even injury. Other protective items are used by infants such as applicant's invention particularized in U.S. patent application Ser. No. 11/209,728 filed Aug. 24, 2005.

Furthermore protective gaiters have heretofore been utilized which extend from the wearers instep to the knee region. More particularly the gaiter extends to above the knee. For example, U.S. Pat. No. 4,001,953 relates to a protective gaiter which extends from a wearers instep to just below the knee. A portion of the gaiter forms a completely closed pocket which contains an energy absorbing pad. Furthermore U.S. Pat. No. 5,010,597 relates to a gaiter for bicycling which incorporates an internal pocket capable of holding small items such as keys, change or I.D. when bicycling. Moreover, U.S. Pat. No. 4,035,860 further relates to a protective gaiter.

Other protective pads have also been utilized in the prior art which includes a pad for protecting at least a portion of the human body against injury including at least three hybrid fabric layers as particularized in U.S. Pat. No. 5,395,683.

It is an object of this invention to provide an improved protective garment which can be utilized by individuals and particularly by infants which are prone to bumps and bruises when developing in and from the crawling stage.

The need for protective garments extends not only to gaiters but also to mittens and diapers.

### DISCLOSURE OF INVENTION

It is an aspect of this invention to provide a garment having a cushion material.

It is another aspect of this invention to provide a gaiter with a pocket for a protective pad.

It is another aspect of this invention to provide a protective glove or mitten.

It is yet another aspect of this invention to provide a diaper pad.

Finally it is an aspect of this invention to provide a crib cover.

### BRIEF DESCRIPTION OF DRAWINGS

These and other features and objects of the invention shall now be described in relation to the following drawings.

FIG. 1 is representative view of the gaiter.

FIG. 2 is a top plan view of a glove.

FIG. 3 is a representative view of a diaper with pocket.

FIG. 4 is a top plan view of a self-adhesive diaper pad.

FIG. 5 is a representative view of a crib cover.

### DESCRIPTION OF THE PREFERRED EMBODIMENTS

In the description which follows, like parts are marked throughout the specification and the drawings with the same respective reference numerals. The drawings are not necessarily to scale and in some instances proportions may have been exaggerated in order to more clearly depict certain features of the invention.

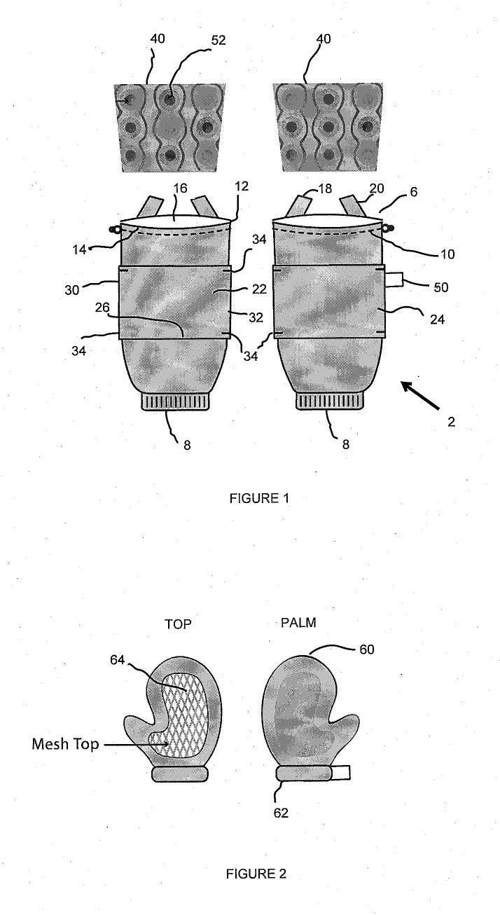

FIG. 1 is a front elevational view of a garment 2 which comprises gaiters or leggings 4 and 6 which are adapted to be attached to the left and right legs of an individual (not shown) or infant.

The gaiters 4 and 6 can be comprised of any suitable material used for clothing. In one embodiment the gaiters 4 and 6 are comprised of tough nylon to protect against slivers, wet surfaces, rough surfaces and dirt particularly when the gaiters are applied to an infant who may be crawling either inside or outside the home. The gaiter can also be made of cotton or other fabric. The gaiters are adapted to be worn over any pant leg or leg of an infant.

Although the invention is described in relation to infants the gaiters may also be worn by any individuals such as gardeners, roofers, floor sanders or the like.

Each of the gaiters includes at the bottom thereof an elasticized sleeve 8 which is adapted to be expanded so as to permit the insertion of an individual's foot there through and to contract the leg or ankle once the leg has been inserted so as to provide a snug fit around the ankle area of the user.

The gaiters 4 and 6 include at the top thereof stitching to provide a circular or circumferential passage 12 adapted to receive a rope or chord 14 or the like.

The chord or rope 14 is located within the passage and extends outwardly there from so as to decrease the size of the opening 16 for receiving the foot of the user. The toggle 18 may then releaseably secure the ends of the chord 14 once in a desired position so as to maintain the desired size of the opening and maintain the securing pressure.

The gaiters 4 and 6 may also include Velcro™ tabs 18 and 20 which can be used as an added means of securing the gaiters to the leg of a user.

The gaiters 4 and 6 include one pocket or enclosure 22 in the vicinity of the knee region. The pocket 22 covers the knee region of the garment 2.

Preferably each pocket 22 is the full width across the knee region of the gaiters 4 and 6. The pocket 22 can be stitched or sewn unto the gaiter in the knee region. The pocket 22 in one embodiment can consist of an extra piece of fabric 24 stitched on top of the gaiters in the vicinity of the knee area. In other words, the pocket is stitched on the top edge 26 and bottom edge 28. The sides 30 and 32 may be open so as to accommodate the insertion of padded material 40. Alternatively the top surface of the pocket may be the top surface of the gaiter.

In one embodiment one of the sides 30 and 32 may be stitched, leaving the other one of the sides 30 and 32 open so as to accommodate the insertion of the padded material 40. Alternatively, both sides 30 and 32 may be open.

It can be seem from FIG. 1 that reinforced stitching 34 can be utilized so that the seams of the sides 30 and 32 between the stitching 34 is open while the sides 30 and 32 are stitched between the reinforcements 34 and top portion 26 and bottom portion 28 respectively. Accordingly the opening to the pocket is slightly smaller than the padding 40 so as to minimize the possibility of accidental removal of the pads particularly when the infant is crawling along a surface such as grass or the like. Accordingly the sewn edges or reinforcements 34 trap the knee pad.

Accordingly the pockets define at least one open side 50 which is utilized to receive the padding.

Any suitable padding can be utilized but in one embodiment neoprene pads having a plurality of elevated bumps or protrusions 52 as shown in FIG. 1 can be utilized. The elevated bumps 52 are utilized for extra cushioning particularly when the infant is crawling outdoors. Alternatively, a neoprene pad 22 could be used which does not have elevated bumps. In one embodiment a 5 millimetre thick neoprene pad 22 could be used.

The side vents or opening 50 on the pockets 22 and 24 can be used to remove the padding for easy washing.

In another embodiment of the invention 22 the pocket could be sewn inside the gaiter or the pocket 22 sewn shut with the padded material inside the sewn pocket. Also, the neoprene pad 22 could be sewn into the leg of the gaiter either inside or outside. In other words the neoprene pad could be sewn on top of the fabric of the gaiter leg or onto the fabric inside the gaiter leg

Moreover FIG. 2 illustrates the utilization of neoprene on mittens that can be used for toddlers which are crawling particularly outdoors.

The mittens or gloves 60 can be comprised of any suitable material such as fleece, cotton, treated cotton or other fabric, and have padded palms to protect against slivers, wet surfaces, rough surfaces and dirt while crawling outdoors. The mittens also include a Velcro™ closure 62 as shown so as to minimize the possibility of accidental removal of the mittens.

The neoprene padding can be inserted inside of the mitten. In one embodiment the inside of the Mitten includes a pocket adapted to receive the cushy neoprene padding. In other embodiment the neoprene padding may include an adhesive which is adapted to stick to the outside of the mitten. Alternatively an outside pocket may be utilized. Also the mitten can be with neoprene or without. As a further alternative a rubber treated material could be sewn on the outside of the mitten 60 for traction, waterproof surface, durable surface and to protect against slivers or sharp objects from penetrating the mitten. The rubber is a thin layer of rubber sewn across the bottom of the mitt, to cover the entire bottom surface.

Furthermore the mitten 60 may also include a meshed region 64 to permit ventilation between the outside and inside of the mitten so as to keep the infant cool.

Accordingly the gaiters and mittens as described above may be utilized by individuals such as infants or toddlers who are crawling outside so as to minimize the possibility of scraps and bruises as the protective padding will absorb the bumps and bruises as well as protect against slivers and splinters.

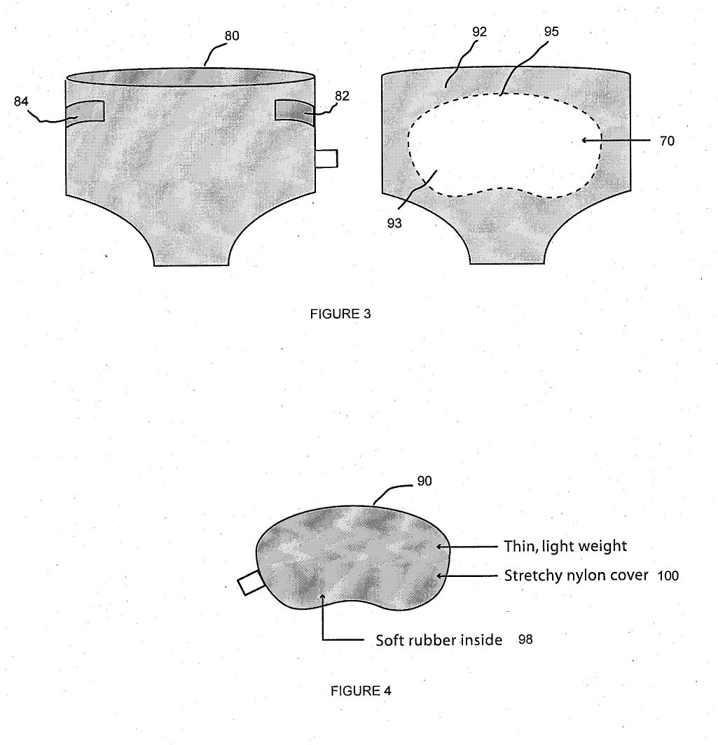

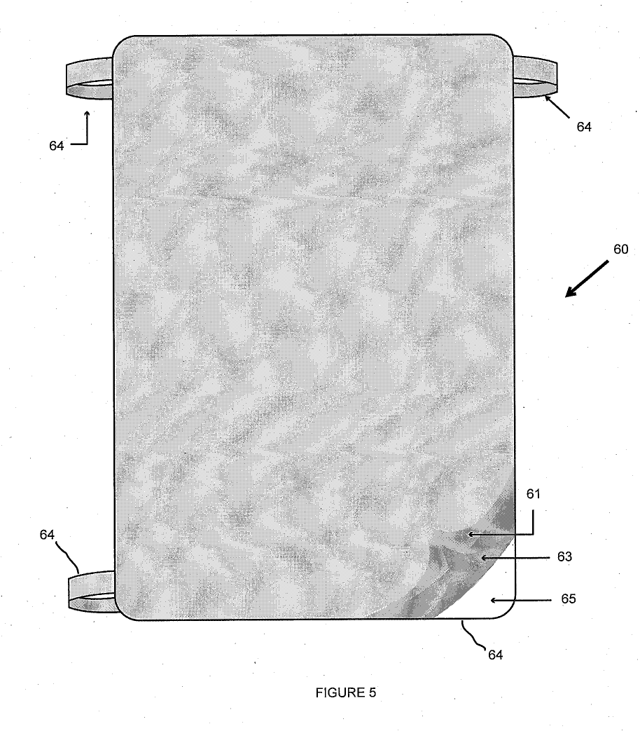

Furthermore the protective pads as described above may also be utilized in association with diapers as shown in FIGS. 3 and 4.

It is not unusual for infants to accidentally fall unto the backside when attempting to walk. Accordingly FIG. 3 shows the use of a protective pad 70 in association with diaper pants 80 having Velcro™ straps or tabs 82 and 84. The diaper pants 80 can comprise of any suitable material as used in the prior art. The diaper pants have a neoprene insert that slides into its own “burn pocket.” The pad may be inserted into the “burn” pocket and pulled over the babies diaper and the garment is ready for use.

Alternatively diaper 80 may include a pocket adapted to receive a protective pad 90. Preferably the pocket 92 can be disposed on the exterior of the diaper 80 as shown in FIG. 3 and may have an opening 93 along the side thereof as previously described. Alternatively, the pocket 92 may have an opening at the top 95. Preferably the pocket opens exteriorly of the diaper although it can also be located in interiorly of the diaper. Also the pocket can open on the diaper pad or the pad can be sewn in permanently.

As a further alternative the protective diaper pads 90 may include a self-adhesive along one surface 97 thereof to adhere to the bottom of any diaper. The protective pad may be comprised of neoprene or any other thin light weight material. The sticky adhesive adheres to the diaper. In one embodiment the diaper pad 90 includes a soft rubber inside 98 with a stretchy nylon cover 100. Accordingly the pad 90 as disclosed in FIG. 4 illustrates a self-adhesive diaper pad.

Finally, FIG. 5 illustrates a crib cover which can be utilized to relieve the parent from making up the whole bed of an infant in the middle of the night.

In particular the crib cover 60 comprises a soft flannel surface 62 sized so as to fit within the bottom of the bed. The corners of the cover 60 can include tabs 64 which can comprise Velcro™ or other fastening means to wrap around the crib slats of an infants bed.

The crib cover 60 can include a soft flannel layer 61 at the top thereof as well as a second soft padding material 63 such as neoprene or the like. Furthermore a third layer comprising of plastic 65 can be disposed at the bottom thereof. The crib pad can also consist of two layers, namely atop sheet layer and a plastic layer and can be sewn together.

Accordingly the plastic bottom portion provides rigidity to the cover while the soft cushy padding provides resilient cushioning to an infant so as to aid in providing a soft sleeping surface. Moreover the soft flannel surface further enhances the comfort of the crib cover. The layers may be bundled together by a draw string carried by the outside periphery of the flannel layer so that the layers can be separated and washed.

Accordingly if the baby wakes in the middle of the night due to wetness the parent does not need to make up the whole bed as it will only be necessary to change the diaper and pajamas of the baby and replace the crib cover in an easy and efficient manner.

In one embodiment the crib cover lays flat on the top of the wet sheet. The baby is put on top of the crib cover and then the Velcro™ tabs are fastened to the crib. In other words, one can change the sleeping surface while holding the baby in ones arms.

Alternatively, the crib cover can have sides that go over the mattress and then taken off when wet.

The inventions described herein are particularly useful for young children who crawl on the floor as the pocket opening is generally disposed on the lateral side of the pocket and the top and bottoms of the pocket retain the pad. However, the inventions described herein can also be used by adults when kneeling during gardening or other functions requiring kneeling or the like.

Various embodiments of the invention have now been described in detail. Since changes in and/or additions to the above-described best mode may be made without departing from the nature, spirit or scope of the invention, the invention is not to be limited to said details.

I claim:

1. A garment having a cushion material.

2. A garment as claimed in claim 1 wherein said cushion material is neoprene.

3. A garment as claimed in claim 2 wherein said garment comprises a gaiter.

4. A garment as claimed in claim 3 wherein said gaiter includes a pocket for said, protective pad.

5. A garment as claimed in claim 4 wherein said pocket includes a side opening adapted to receive said protective pad.

6. A garment as claimed in claim 5 wherein said protective pad comprises neoprene.

7. A garment as clamed in claim 2 wherein said neoprene is sewn into the top of the garment either outside or inside the gaiter leg

8. A garment as claimed in claim 4 wherein said pocket is disposed outside the garment.

9. A garment as claimed in claim 4 wherein said pocket is disposed inside the garment.

10. A garment as claimed in claim 4 wherein said pocket is sewn shut.

11. A garment as claimed in claim 4 wherein aid pocket is open.

12. A garment as claimed in claim 1 comprising a mitten.

13. A garment as claimed in claim 12 wherein said mitten includes a pocket for said protective pad.

14. A garment as claimed in claim 13 wherein said protective pad comprises neoprene.

15. A garment as claimed in claim 14 wherein said mitten comprises a mesh opening.

16. A garment as claimed in claim 1 comprising a diaper.

17. A garment as claimed in claim 16 wherein said diaper includes a neoprene pad.

18. A garment as claimed in claim 17 wherein the neoprene pad comprises a self-adhesive material for removably securing to said diaper.

19. A bed cover for a crib having a plastic layer, an intermediate padding layer and a flannel layer, or a top sheet layer and a plastic layer sewn together.

20. A crib cover as claimed in claim 14 having fastening means at the corners thereof for fastening to said crib slats.
